(19) United States
(12) Patent Application Publication
(10) Pub. No.: US 2007/0027408 A1
(43) Pub. Date: Feb. 1, 2007

(54) Anatomical Feature Tracking and Monitoring System

(75) Inventors: Loretta A. Fitzgerald, Collegeville, PA (US); Lisa T. O'Neill, West Chester, PA (US)

Correspondence Address: ALEXANDER J BURKE, 3 GLENSIDE TERRACE, UPPER MONTCLAIR, NJ, 07043, US

(73) Assignee: SIEMENS MEDICAL SOLUTIONS HEALTH SERVICES CORPORATION, MALVERN, PA (US)

(21) Appl. No.: 11/420,097

(22) Filed: May 24, 2006

Related U.S. Application Data
(60) Provisional application No. 60/697,116, filed on Jul. 7, 2005.

(51) Int. Cl. A61B 5/103

(52) U.S. Cl. 600/587

(57) ABSTRACT

A system according to invention principles automatically calculates anatomical feature (e.g., tumor, body part, organ) size electronically to provide a clinician with useful information when assessing the tumor response during treatment. A system for monitoring change in patient anatomical features, includes a volumetric analyzer for automatically analyzing data derived from an imaging unit that represents a three dimensional view of a patient anatomical feature in order to determine spatial characteristics of the anatomical feature. A comparison processor automatically compares a first set of spatial characteristics of a patient anatomical feature derived at a first time with a second set of corresponding spatial characteristics of the patient anatomical feature derived at a subsequent second time, to provide change data representing change in spatial characteristics of the patient anatomical feature over a period of time. A repository stores change data for access by a user.

This is a non-provisional application of provisional application Ser. No. 60/697,116 by L. A. Fitzgeraid et al. filed Jul. 7, 2005.

### FIELD OF THE INVENTION

This invention concerns a system for monitoring change in patient anatomical features involving determining change in spatial characteristics of patient anatomical features over a period of time.

### BACKGROUND OF THE INVENTION

The existing method of determination of tumor size is typically a manual process that requires a healthcare worker to document the acquired measurements detailing size of a tumor over a period of time. The measurements acquired over a period of time are compared and used to calculate changes in tumor size including volume, and lengths in three dimensions. Tie changes in tumor size over time are used to determine a response of a tumor to treatment. A healthcare worker measures a tumor from diagnostic images, derived from an imaging modality device such as an MRI or Ultrasound device, for example, and compares the measurements to previous measurments made of the tumor recorded using a paper chart. This process is error prone and labor intensive. A system according to invention principles addresses these deficiencies and related problems.

### SUMMARY OF THE INVENTION

A system according to invention principles automatically calculates anatomical feature (e.g., tumor, body part, organ) size electronically and stores this infornation for further evaluation and enables a clinician to compare previous measurements and calculate change in feature size. A system for monitoring change in patient anatomical features, includes a volumetric analyzer for automatically analyzing data derived from an imaging unit that represents a three dimensional view of a patient anatomical feature in order to determine spatial characteristics of the anatomical feature. A comparison processor automatically compares a first set of spatial characteristics of a patient anatomical feature derived at a first time with a second set of corresponding spatial characteristics of the patient anatomical feature derived at a subsequent second time, to provide change data representing change in spatial characteristics of the patient anatomical feature over a period of time. A repository stores change data for access by a user.

### BRIEF DESCRIPTION OF THE DRAWING

FIG. 1 shows a system for monitoring change in patient anatomical features, according to invention principles.

FIG. 2 illustrates a patient medical record indicating chance in a patient anatomical feature over a period of time, according to invention principles.

FIG. 3 shows a flowchart of a process used by a system for monitoring change in patient anatomical features, according to invention principles.

### DETAILED DESCRIPTION OF THE INVENTION

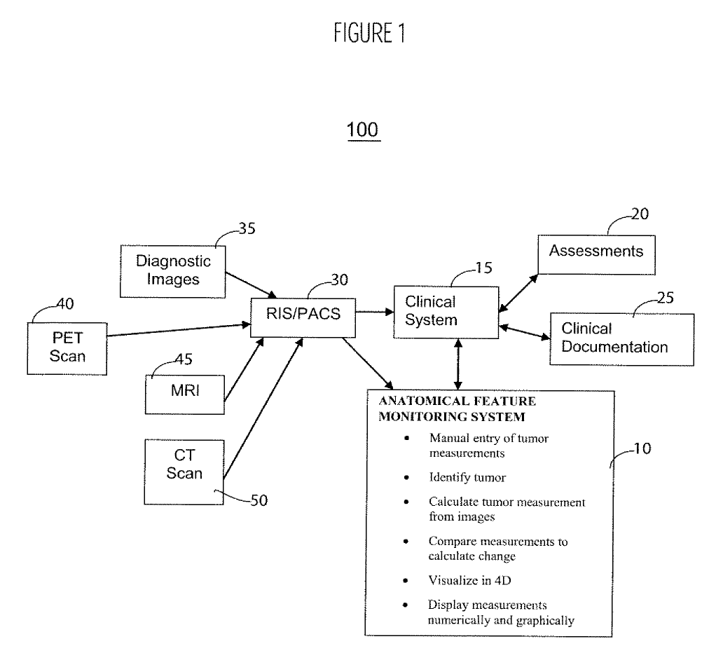

FIG. 1 shows a system for monitoring change in patient anatomical features by automatically calculating tumor size electronically and storing this information for further evaluation. The system enables a clinician to compare previous measurements and calculate change in the tumor size. The system allows a clinician to enter tumor dimensions manually or the system receives them in electronic data form from another system and calculates tumor volume and change in tumor size using previously recorded tumor measurements. The calculation of change in tumor size may be made between measurements entered electronically, manually, or a combination of both. The system provides a user with a numerical display and graphical display of the tumor and change in size. Numerically, the results provided include length, width, height, volume of change and percentage volume change. Graphically, the system provides a user with a display of the tumor as it was originally measured and a visual indication of the change in volume over a period of time. The user is able to advantageously view this information in 4D (3 physical dimensions and over time) to analyze the change in size and estimate ramifications of such a change on surrounding anatomy over a period of time.

The system reduces the risk of error when calculating tumor size and manually calculates change in tumor size, for example, by performing these functions electronically. The system also eliminates the need for a clinician to search for a paper chart when performing a tumor measurement comparison. The system provides an electronic means to calculate the size of a tumor from diagnostic images and compare these measurements to previous measurements and also provides a user with a means to visualize a tumor in four dimensions (4D, in relation to surrounding structures and time), and provides a clinician with additional information to assess tumor progression or regression. The derived information is available in an electronic form for access by other clinicians treating the patient and also allows a user to add this information to other available electronic documentation, such as flowsheets, assessments, patient medical records and dictated reports in numerical and graphical representations.

An executable application comprises code or machine readable instruction for implementing predetermined functions including those of an operating system, healthcare infornation system or other information processing system, for example, in response user command or input. An executable procedure is a segment of code (machine readable instruction), sub-routine, or other distinct section of code or portion of an executable application for performing one or more particular processes and may include performing operations on received input parameters (or in response to received input parameters) and provide resulting output parameters. A processor as used herein is a device and/or set of machine-readable instructions for performing tasks. A processor comprises any one or combination of, hardware, firmware, and/or software. A processor acts upon information by manipulating, analyzing, modifying, converting or transmitting information for use by an executable procedure or an information device, and/or by routing the information to an output device. A processor may use or comprise the capabilities of a controller or microprocessor, for example. A display processor or generator is a known element comprising electronic circuitry or software or a combination of both for generating display images or portions thereof. A user interface comprises one or more display images enabling user interaction with a processor or other device.

FIG. 1 shows system 100 for monitoring change in patient anatomical features by automatically calculating tumor size electronically and storing this information for further evaluation. In system 100 a RIS—PACS (Radiology Information System—Picture Archiving and Computer System) 30 acquires and stores data representing diagnostic medical images 35 from different imaging modality devices including PET scan device 40, MRI device 45 and CT scan device 50. Other modality imaging devices (not shown) may include, X-ray, Ultrasound and other devices. Data representing one or more medical images 35 stored in PACs system 30 is accessed by clinical information system (or hospital information system) 15 and by anatomical feature monitoring system 10. Clinical information system 15 acquires data comprising patient clinical assessments 20 entered by a clinician such as diagnoses, progress reports, examination notes, for example as well as clinical documentation 25 such as treatment plans, consents and treatment protocols.

System 100 provides a user with electronic access to diagnostic images 35. System 100 supports user identification of anatomical features such as a tumor, organ, body part or portion thereof. System 100 also supports automatic identification of such anatomical features. Anatomical feature monitoring system 10 receives data representing spatial characteristics of an anatomical feature comprising both linear dimensions and volume of the feature. The spatial characteristics may be manual measurements or automatically derived. A clinician may manually measure an anatomical feature on various diagnostic images such as CT, MRI, or flat panel X-ray images. In another technique a user manually traces the boundary of a feature or areas of interest from cross sectional images generated from a CT scan or MRI and the volume is calculated by a computer using a pixel counting method, for example. More advanced known techniques that may be employed may be point-counting stereology based on a fixed-grid stereologic volumetry approach, a random-grid stereologic volumetry approach, or a grid-point counting stereological method. System 10 is adaptable to use any known feature measurement methods.

Anatomical feature monitoring system 10 includes a volumetric analyzer for automatically analyzing data derived from an imaging unit and representing a three dimensional view of a patient anatomical feature, to determine spatial characteristics of the anatomical feature. The imaging unit comprises an imaging modality including at least one of, (a) a CT scan device and (b) an MRI device, (c) an Ultrasound device and (d) a radiological scanning device. The volumetric analyzer analyzes data representing a three dimensional view of a patient anatomical feature in multiple activity modes, to determine spatial characteristics of the anatomical feature in the multiple activity modes. The activity modes include at least one of, ambulatory and non-ambulatory, different heart cycles and different respiratory phases. A user of system 100 and anatomical feature monitoring system 10 is able to identify upper and lower margins of a tumor via cursor manipulation and in-between areas of the tumor and to add this information as annotation to an image data file, for example.

A user is also able to add annotation to an image data file to identify the type of imaging modality device providing an image from which tumor measurements are derived such as a CT scan device, for example. A user employs feature monitoring system 10 to select a CT slice and identify an upper most border of a tumor and identify the tumor. Similarly, a user identifies the lower tumor border, and CT slices in between. In response to a tumor volume being identified, a user initiates determination of tumor spatial characteristics (such as linear dimensional measurements and volume measurements) by feature monitoring system 10. An anatomical feature spatial characteristic may also comprise an enclosed or interior dimension or volume, e.g., ventricle interior volume, artery or vain diameter, or other interior dimension or volume. The linear dimensional measurements are used by system 10 to calculate tumor volume. The spatial characteristics are stored in the system 10 and accessible to add to any clinical documentation.

A comparison processor in anatomical feature monitoring system 10 automatically compares a first set of spatial characteristics of a patient anatomical feature derived at a first time with a second set of corresponding spatial characteristics of the patient anatomical feature derived at a subsequent second time and stored in memory in unit 10 or within RIS-PACs system 30 or clinical information system 15. The comparison processor provides change data representing change in spatial characteristics of the patient anatomical feature over a period of time. The comparison processor in one embodiment, compares a first set of spatial characteristics of a patient anatomical feature in a first activity mode derived at a first time with a second set of corresponding spatial characteristics of the patient anatomical feature in the first activity mode derived at a subsequent second time, to provide the change data. Feature monitoring system 10 stores the change data for access by a user in a repository in unit 10 or elsewhere in system 100.

A display processor in feature monitoring system 10 initiates generation of data representing an image including the patient anatomical feature derived at the first time and the patient anatomical feature derived at the subsequent second time and the change data. The patient anatomical features derived at the first and second time are displayed using different visual display characteristics for individual feature identification. A documentation processor in system 10 automatically incorporates the change data in a document comprising a medical report, a flowsheet, a treatment plan, a patient assessment or a dictated report. The documentation processor automatically incorporates an image of the patient anatomical feature into the document.

A clinician is provided with the ability to select a previously entered tumor (or feature) volume, and to initiate calculation of the difference between current and previous volume measurements. This analysis is performed on one or many previously derived measurements, and generates change in size by millimeters or centimeters, for example and a percentage. A clinician is also provided with the ability to view the change in four Dimensions. A graphical tumor representation provides a visual indicator of the previous tumor measurements and another visual indicator of the present tumor measurements. For example, a clinician refers to previous CT scans and indicates a tumor on multiple CT slices. The clinician selects the most recent CT scans and again identifies the tumor from multiple slices. System 10 calculates the tumor volume from both image studies and provides the measurements to a user in the preferred unit of measure (such as centimeters or millimeters), and the volume and percentage of change. The clinical user is able to view the previous tumor size highlighted in one color while the most recent tumor size is highlighted in another color and has the ability to select the color display using a configuration processor in system 10. A clinician using system 10 is provided with an ability to view a tumor in relation to surrounding anatomical structures provided by a diagnostic image (CT scan, MRI, etc.). An image view of a tumor in four dimensions in relation to sensitive anatomical structures, for example, is of significant benefit to a clinician in assessing patient condition and assessing the progress of tumor growth or reduction in size.

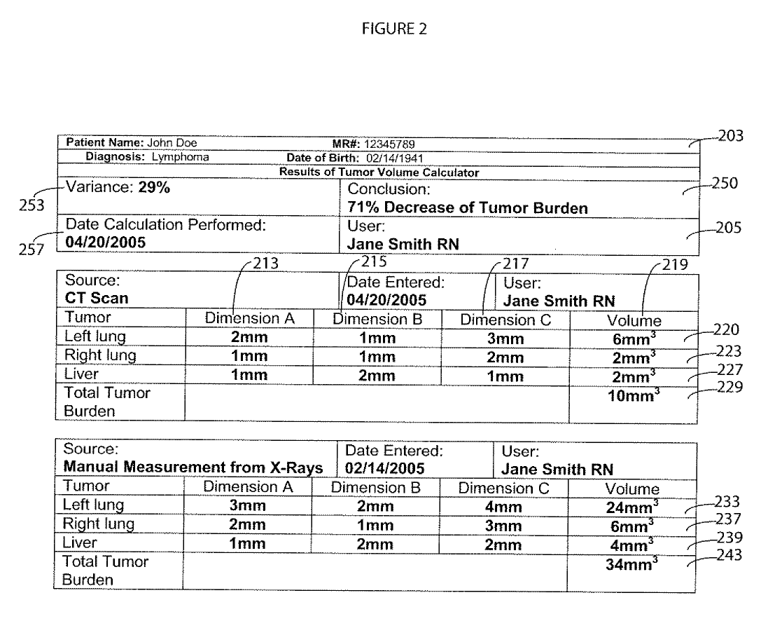

FIG. 2 illustrates a patient medical record generated by feature monitoring system 10 indicating change in anatomical features of a patient identified in row 203 over a period of time. The record is made based on measurements initiated by a user (Jane Smith) identified in row 205 and made using medical images of the patient on two separate occasions (Apr. 20, 2005 and Feb. 14, 2005). Measurements in three dimensions listed in columns 213, 215 and 217 of tumors in the left lung, right lung and liver are presented in rows 220, 223 and 227 for images made on Apr. 20, 2005 and in rows 233, 237 and 239 for images made on Feb. 14, 2005. Feature monitoring system 10 calculates volumes of the tumors for the two different occasions as indicated in column 219 as well as a total tumor volume indicated in rows 229 and 243 for the two different occasions. System 10 automatically compares the first set of tumor spatial characteristics derived on Feb. 14, 2005 with the second set of corresponding tumor spatial characteristics derived on Apr. 20, 2005, to provide change data representing change in spatial characteristics of the tumors in response to acquisition of the second set of spatial characteristics (on calculation date Apr. 20, 2005, item 257). System 10 automatically calculates decrease in total tumor volume 250 and variance 253. The variance is the second total tumor volume 10 divided by the first total tumor volume 34 (variance is 29%). Therefore, the patient had a 71% decrease in tumor volume over the period concerned.

System 10 electronically calculates and stores tumor measurements in contrast to the manual process of existing systems. This reduces the risk of human error and ensures the electronically captured information is concurrently available to multiple clinicians responsible for the care of a patient and that treatment is not delayed while a clinician tries to find a paper chart. The system may document measurements taken of anatomical features using known feature measurement methods and calculates the change in a feature over time and provide a means for a user to display this information in various views such as trending, graphical, and tabular display views.

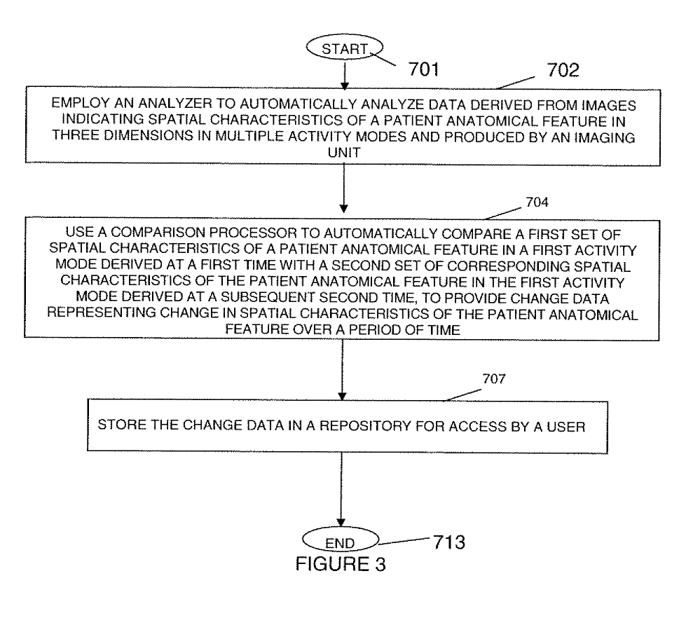

FIG. 3 shows a flowchart of a process used by feature monitoring system 10 for monitoring change in patient anatomical features. In step 702 following the start at step 701, an analyzer in feature monitoring system 10 automatically analyzes data derived from images indicating spatial characteristics of a patient anatomical feature in three dimensions in a plurality of activity modes and produced by an imaging unit. The activity modes include ambulatory or non-ambulatory (e.g., simply lying prone in a scanning device), different heart cycles and different respiratory phases. In step 704, comparison processor in feature monitoring system 10 automatically compares a first set of spatial characteristics of a patient anatomical feature in a first activity mode derived at a first time with a second set of corresponding spatial characteristics of the patient anatomical feature in the first activity mode derived at a subsequent second time, to provide change data representing change in spatial characteristics of the patient anatomical feature over a period of time. System 10 in step 707, stores the change data in a repository for access by a user. The process of FIG. 3 terminates at step 713.

The systems and processes presented in FIGS. 1-3 are not exclusive. Other systems and processes may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A system according to invention principles is applicable to any field in which comparisons need to be made to change in item shape or size, such as adding an addition onto a home, for example. Further, any of the functions provided in the system of FIG. 1 may be implemented in hardware, software or a combination of both and may reside on one or more processing devices located at any location of a network linking the FIG. 1 elements or another linked network including another intra-net or the Internet.

What is claimed is:

1. A system for monitoring change in patient anatomical features, comprising:
a volumetric analyzer for automatically analyzing data derived from an imaging unit and representing a three dimensional view of a patient anatomical feature, to determine spatial characteristics of said anatomical feature;
a comparison processor for automatically comparing a first set of spatial characteristics of a patient anatomical feature derived at a first time with a second set of corresponding spatial characteristics of said patient anatomical feature derived at a subsequent second time, to provide change data representing change in spatial characteristics of said patient anatomical feature over a period of time; and
a repository for storing said change data for access by a user.

2. A system according to claim 1, wherein
said imaging unit comprises an imaging modality including at least one of, (a) a CT scan device and (b) an MRI device, (c) an Ultrasound device and (d) a radiological scanning device.

3. A system according to claim 1, wherein
said spatial characteristics comprise at least one of, (a) a linear dimension and (b) a volume.

4. A system according to claim 1, wherein
said spatial characteristics comprise both linear dimensions and volume.

5. A system according to claim 1, wherein
said volumetric analyzer analyzes data representing a three dimensional view of a patient anatomical feature in a plurality of activity modes, to determine spatial characteristics of said anatomical feature in said plurality of activity modes; and
said comparison processor compares a first set of spatial characteristics of a patient anatomical feature in a first activity mode derived at a first time with a second set of corresponding spatial characteristics of said patient anatomical feature in said first activity mode derived at a subsequent second time, to provide change data representing change in spatial characteristics of said patient anatomical feature over a period of time.

6. A system according to claim 5, wherein
said activity modes include at least one of, (a) ambulatory and non-ambulatory, (b) different heart cycles and (c) different respiratory phases.

7. A system according to claim 1, wherein
said anatomical feature is a tumor.

8. A system according to claim 1, including
a display processor for initiating generation of data representing an image including said patient anatomical feature derived at said first time and said patient anatomical feature derived at said subsequent second time and said change data.

9. A system according to claim 8, including
said patient anatomical features derived at said first and second time are displayed using different visual display characteristics for individual feature identification.

10. A system according to claim 1, including
a documentation processor for incorporating said change data in a document comprising at least one of, (a) a medical report, (b) a flowsheet, (c) a treatment plan, (d) a patient assessment and (e) a dictated report.

11. A system according to claim 10, wherein
said documentation processor incorporates an image of said patient anatomical feature into said document.

12. A system according to claim 1, including
a display processor for initiating generation of data representing an image including change data representing said change in spatial characteristics of said patient anatomical feature over a period of time in at least one of, (a) a trend indicative display, (b) a graphical display and (c) a tabular display.

13. A system for monitoring change in patient anatomical features, comprising:
an analyzer for automatically analyzing data derived from images indicating spatial characteristics of a patient anatomical feature in three dimensions and produced by an imaging unit;
a comparison processor for automatically comparing a first set of spatial characteristics of a patient anatomical feature derived at a first time with a second set of corresponding spatial characteristics of said patient anatomical feature derived at a subsequent second time, to provide change data representing change in spatial characteristics of said patient anatomical feature over a period of time; and
a repository for storing said change data for access by a user.

14. A system according to claim 13, wherein
said imaging unit comprises an imaging modality including at least one of, (a) a CT scan device and (b) an MRI device, (c) an Ultrasound device and (d) a radiological scanning device.

15. A system according to claim 13, wherein
said spatial characteristics comprise at least one of, (a) a linear dimension and (b) a volume.

16. A system for monitoring chance in patient anatomical features, comprising:
an analyzer for automatically analyzing data derived from images indicating spatial characteristics of a patient anatomical feature in three dimensions in a plurality of activity modes and produced by an imaging unit;
a comparison processor for automatically comparing a first set of spatial characteristics of a patient anatomical feature in a first activity mode derived at a first time with a second set of corresponding spatial characteristics of said patient anatomical feature in said first activity mode derived at a subsequent second time, to provide change data representing change in spatial characteristics of said patient anatomical feature over a period of time; and
a repository for storing said change data for access by a user.

17. A system according to claim 16, wherein
said activity modes include at least one of, (a) ambulatory and non-ambulatory, (b) different heart cycles and (c) different respiratory phases.
